(19) United States
(12) Patent Application Publication
(10) Pub. No.: US 2024/0197967 A1
(43) Pub. Date: Jun. 20, 2024

(54) MEDICAL DEVICES FORMED FROM HIGH PURITY POLYURETHANE

(71) Applicant: Boston Scientific Scimed, Inc., Maple Grove, MN (US)

(72) Inventors: Yen-Hao Hsu, Shrewsbury, MA (US); Joseph Thomas Delaney, JR., Minneapolis, MN (US)

(73) Assignee: Boston Scientific Scimed, Inc., Maple Grove, MN (US)

(21) Appl. No.: 18/516,494

(22) Filed: Nov. 21, 2023

Related U.S. Application Data
(60) Provisional application No. 63/428,008, filed on Nov. 25, 2022.

(51) Int. Cl. A61L 29/06; B33Y 70/00; C08G 18/28; C08L 77/12

(52) U.S. Cl. CPC A61L 29/06; B33Y 70/00; C08G 18/2895; C08L 77/12; C08L 2203/02

(57) ABSTRACT

In some aspects, the present disclosure pertains to medical devices, at least a portion of which comprises a polymeric region comprising a poly(urethane amide) that comprises urethane (—O—CO—NH—) groups, and amide (—CO—NH—) groups. In other aspects, the present disclosure pertains to 3-D printing processes that comprise reacting (a) a urethane-based acrylamide reactant that comprises a residue of a C₄-C₁₀ hydroxyalkyl acrylamide and a diisocyanate residue selected from a C₆-C₂₀ aromatic diisocyanate residue, a C₄-C₂₀ aliphatic diisocyanate residue, and a C₆-C₂₀ cycloaliphatic diisocyanate residue and (b) a multi-arm polymer comprising 3 or more polymer arms and three or more terminal groups selected from thiol groups, amino groups and hydroxyl groups.

### CROSS-REFERENCE TO RELATED APPLICATIONS

This application claims the benefit of U.S. Provisional Patent Application Ser. No. 63/428,008 filed on Nov. 25, 2022, the disclosure of which is incorporated herein by reference.

### FIELD

The present disclosure pertains to medical devices that are formed using novel polyurethane compositions and precursors thereof.

### BACKGROUND

Long-term implantable devices, including catheters, artificial polymer discs, and coatings, have been widely manufactured in the medical device industry using medical-grade polyurethanes. Polyurethanes are made by reactions between polyols with two or more reactive hydroxyl groups per molecule (e.g., short chain and polymeric diols, triols and higher polyols) and polyisocyanates that have two or more isocyanate groups per molecule (e.g., aliphatic, cycloaliphatic or aromatic diisocyanates, triisocyanates and polyisocyanates), in conjunction with a tin catalyst. For instance, a diisocyanate reacts with a diol according to the following scheme:

O═C═N—R—N═C═O+HO—R′—OH→ [symbol] CO—NH—R—NH—CO—O—R′O [symbol] ₙ

in which n is an integer, R is an organic moiety, such as an aliphatic, cycloaliphatic or aromatic moiety, and R′ is also an organic moiety, such as an aliphatic moiety or a polymeric moiety. The mechanical properties of polyurethanes such as hardness and stretchability can be broadly tuned by varying weight percentages of hard segments (e.g., aromatic diisocyanates) and soft segments (e.g., hydroxyl-terminated polymers).

However, isocyanate (—N═C═O) is an extremely water-sensitive functional group, which can be converted to primary amine in the presence of water, which will further react with free isocyanate to produce urea (—NH—CO—NH—) linkage groups. Furthermore, current formulations of polyurethanes using a slight excess of diisocyanate for reducing water content purposes, which will result in urea linkages, causing unpredictable variations in molecular weight, thermal properties and mechanical properties. This issue has created particular difficulties in quality control associated with extrusion processes in medical device manufacturing.

For these reasons, a novel high purity polyurethane formulation would be desirable, which has no side reactions leading to formation of undesirable species such as urea groups, is biocompatible, is readily scalable, has thermal and mechanical properties that are similar to current polyurethanes, and can act as a replacement in medical devices such as leads and catheters that have cracking concerns after implantation.

### SUMMARY

In various embodiments, the present disclosure pertains to medical devices, at least a portion of which comprise a polymeric region comprising a poly(urethane amide) that comprises urethane (—O—CO—NH—) groups and amide (—CO—NH—) groups.

In some embodiments, which are applicable to any of the preceding embodiments, the poly(urethane amide) further comprises amine (—NH—) linkages.

In some embodiments, which are applicable to any of the preceding embodiments, the poly(urethane amide) further comprises thioether (—S—) linkages.

In some embodiments, which are applicable to any of the preceding embodiments, the poly(urethane amide) further comprises ether (—O—) linkages.

In some embodiments, which are applicable to any of the preceding embodiments, the poly(urethane amide) comprises (a) one or more diisocyanate residues selected from C₆-C₂₀ aromatic diisocyanate residues, C₄-C₂₀ aliphatic diisocyanate residues, and C₆-C₂₀ cycloaliphatic diisocyanate residues, (b) residues of a C₄-C₁₀ hydroxyalkyl acrylamide, and (c) residues of a polythiol, a polyamine, or a polyol.

For example, the one or more diisocyanate residues may be selected from residues of toluene diisocyanate (TDI), methylene diphenyl diisocyanate (MDI), naphthalene diisocyanate (NDI), toluidine diisocyanate, including 1,6-hexamethylene diisocyanate (HDI), tetramethylene diisocyanate, 1-isocyanato-3-isocyanatomethyl-3,5,5-trimethyl-cyclohexane (isophorone diisocyanate), and 4,4′-diisocyanato dicyclohexylmethane (H₁₂MDI).

For example, the polythiol may be selected from thiol terminated C₂-C₂₀ aliphatic molecules, thiol terminated polyethers, thiol terminated polycarbonates, thiol terminated poly-epsilon-caprolactones, thiol terminated polyolefins, thiol terminated polyesters, thiol terminated polyoxazolines and thiol terminated polyvinylpyrrolidone.

For example, the polyol may be selected from hydroxyl terminated C₂-C₂₀ aliphatic molecules, hydroxyl terminated polyethers, hydroxyl terminated polycarbonates, hydroxyl terminated poly-epsilon-caprolactones, hydroxyl terminated polyolefins, hydroxyl terminated polyesters, hydroxyl terminated polyoxazolines and hydroxyl terminated polyvinylpyrrolidone.

For example, the polyamine may be selected from amino terminated C₂-C₂₀ aliphatic molecules, amino terminated polyethers, amino terminated polycarbonates, amino terminated poly-epsilon-caprolactones, amino terminated polyesters, amino terminated polyoxazolines and amino terminated poly vinyl pyrrolidoneamino terminated polyolefins, amino terminated polyesters, amino terminated polyoxazolines and amino terminated poly vinyl pyrrolidone.

In some embodiments, which are applicable to any of the preceding embodiments, the poly(urethane amide) is of one of the following formulas (I), (II), or (III):

[symbol]

S—CH₂—CH₂—CO—NH—(CH₂)ₘ—O—CO—NH—R—NH—CO—O—(CH₂)ₘ—NH—CO—CH₂—CH₂—S—R′ [symbol] ₙ (I)

[symbol]

NH—CH₂—CH₂—CO—NH—(CH₂)ₘ—O—CO—NH—R—NH—CO—O—(CH₂)ₘ—NH—CO—CH₂—CH₂—NH—R′ [symbol] ₙ (II)

[symbol]

O—CH₂—CH₂—CO—NH—(CH₂)ₘ—O—CO—NH—R—NH—CO—O—(CH₂)ₘ—NH—CO—CH₂—CH₂—O—R′ [symbol] ₙ (III)

wherein m is an integer ranging from 1 to 20, n is an integer ranging from 1 to 1000, R is selected from C₁-C₂₀ aliphatic moieties, C₆-C₂₀ aromatic moieties, and C₆-C₂₀ cycloaliphatic moieties, and wherein R′ is selected from C₁-C₂₀ aliphatic moieties, polyether moieties, polycarbonate moieties, poly-epsilon-caprolactone moieties, polyolefin moieties polyester moieties, polyoxazoline moieties and polyvinyl pyrrolidone moieties.

In some embodiments, the poly(urethane amide) of the formula (I) is formed by reaction between H₂C═C—CO—NH—(CH₂)ₘ—O—CO—NH—R—NH—CO—O—(CH₂)ₘ—NH—CO—C═CH₂ and HS—R′—SH.

In some embodiments, the poly(urethane amide) of the formula (II) is formed by reaction between H₂C═C—CO—NH—(CH₂)ₘ—O—CO—NH—R—NH—CO—O—(CH₂)ₘ—NH—CO—C═CH₂ and H₂N—R′—NH₂.

In some embodiments, the poly(urethane amide) of the formula (III) is formed by reaction between H₂C═C—CO—NH—(CH₂)ₘ—O—CO—NH—R—NH—CO—O—(CH₂)ₘ—NH—CO—C═CH₂ and HO—R′—OH.

In some embodiments, which are applicable to any of the preceding embodiments, R is selected from benzene, toluene, diphenyl methylene, naphthalene, hexamethylene, tetramethylene, isophorone and dicyclohexyl methylene moieties.

In some embodiments, which are applicable to any of the preceding embodiments, the polymeric region corresponds to an entirety of the medical device, a medical device coating, or a medical device component.

In some embodiments, which are applicable to any of the preceding embodiments, the polymeric region further comprises a therapeutic agent.

In some embodiments, which are applicable to any of the preceding embodiments, the polymeric region further comprises an additive.

Other embodiments of the present disclosure pertain to method of making a medical device comprising:
(a) performing a Michael addition reaction between H₂C═C—CO—NH—(CH₂)ₘ—O—CO—NH—R—NH—CO—O—(CH₂)ₘ—NH—CO—C═CH₂ and HS—R′—SH to form a poly(urethane amide of the formula (I):

[symbol]

S—CH₂—CH₂—CO—NH—(CH₂)ₘ—O—CO—NH—R—NH—CO—O—(CH₂)ₘ—NH—CO—CH₂—CH₂—S—R′ [symbol] ₙ (I);
(b) performing a Michael addition reaction between H₂C═C—CO—NH—(CH₂)ₘ—O—CO—NH—R—NH—CO—O—(CH₂)ₘ—NH—CO—C═CH₂ and H₂N—R′—NH₂ to form a poly(urethane amide of the formula (II):

[symbol]

NH—CH₂—CH₂—CO—NH—(CH₂)ₘ—O—CO—NH—R—NH—CO—O—(CH₂)ₘ—NH—CO—CH₂—CH₂—NH—R′ [symbol] ₙ (II);

or
(c) performing a Michael addition reaction between H₂C═C—CO—NH—(CH₂)ₘ—O—CO—NH—R—NH—CO—O—(CH₂)ₘ—NH—CO—C═CH₂ and HO—R′—OH to form a poly(urethane amide of the formula (III):

[symbol]

O—CH₂—CH₂—CO—NH—(CH₂)ₘ—O—CO—NH—R—NH—CO—O—(CH₂)ₘ—NH—CO—CH₂—CH₂—O—R′ [symbol] ₙ (III);

wherein m is an integer ranging from 1 to 20, n is an integer ranging from 1 to 1000, R is selected from C₂-C₂₀ aliphatic moieties, C₆-C₂₀ aromatic moieties, and C₆-C₂₀ cycloaliphatic moieties, and wherein R′ is selected from C₂-C₂₀ aliphatic moieties, polyether moieties, polycarbonate moieties, poly-epsilon-caprolactone moieties, polyolefin moieties, polyester moieties, polyoxazoline moieties and polyvinyl pyrrolidone moieties.

Other embodiments of the present disclosure pertain to A 3-D printing process for forming a medical device or a medical device component comprising reacting (a) a urethane-based acrylamide reactant that comprises a residue of a C₄-C₁₀ hydroxyalkyl acrylamide and a diisocyanate residue selected from a C₆-C₂₀ aromatic diisocyanate residue, a C₃-C₂₀ aliphatic diisocyanate residue, and a C₆-C₂₀ cycloaliphatic diisocyanate residue and (b) a multi-arm polymer comprising 3 or more polymer arms and three or more terminal groups selected from thiol groups, amino groups and hydroxyl groups.

The above and other aspects, embodiments, features and benefits of the present disclosure will be readily apparent from the following detailed description.

### BRIEF DESCRIPTION OF THE DRAWINGS

FIG. 1 is a schematic illustration of processes for forming poly(urethane amides), in accordance with three embodiments of the present disclosure.

FIG. 2 is a schematic illustration of processes for forming poly(urethane amides), in accordance with three additional embodiments of the present disclosure.

FIG. 3 is a schematic illustration of processes for forming poly(urethane amides), which is useful in conjunction with a 3-D print process, in accordance with further embodiments of the present disclosure.

FIG. 4 is a schematic illustration of processes for forming a further poly(urethane amide), in accordance with an embodiment of the present disclosure.

### DETAILED DESCRIPTION

In the present disclosure, rather than formulating polyurethanes by using diisocyanates, short chain diols, and polyether diols in conjunction with a tin catalyst, a hydroxyalkyl acrylamide molecule, H₂C═C—CO—NH—(CH₂)ₘ—OH, where m ranges from 1 to 20 (i.e., m is 1, 2, 3, 4, 5, 6, 7, 8, 9, 10, 11, 12, 13, 14, 15, 16, 17, 18, 19 or 20), is reacted with a diisocyanate, O═C═N—R—N═C═O, where R is an organic moiety, such as an aliphatic, cycloaliphatic or aromatic moiety, to produce a urethane-based acrylamide, H₂C═C—CO—NH—(CH₂)ₘ—O—CO—NH—R—NH—CO—O—(CH₂)ₘ—NH—CO—C═CH₂.

Particular examples of diisocyanates for forming such urethane-based acrylamides include aromatic diisocyanates such as toluene diisocyanate (TDI), methylene diphenyl diisocyanate (MDI), naphthalene diisocyanate (NDI) and toluidine diisocyanate, and aliphatic and cycloaliphatic diisocyanates such as 1,6-hexamethylene diisocyanate (HDI), tetramethylene diisocyanate, 1-isocyanato-3-isocyanatomethyl-3,5,5-trimethyl-cyclohexane (isophorone diisocyanate, IPDI) and 4,4′-diisocyanato dicyclohexylmethane (H₁₂MDI or hydrogenated MDI).

For instance, in one specific embodiment, N-hydroxyethyl acrylamide (CAS# 7646-67-5) is reacted with 1,6-hexamethylene diisocyanate (CAS# 822-06-0) to produce a urethane-based acrylamide as follows:

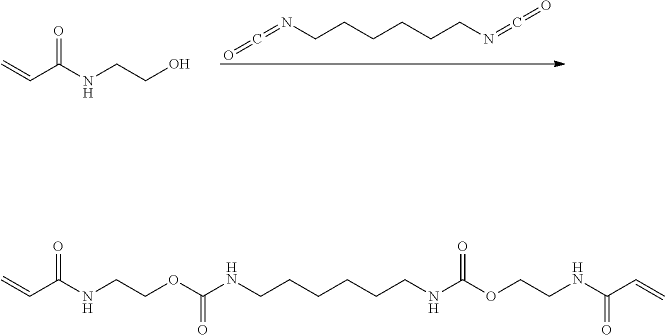

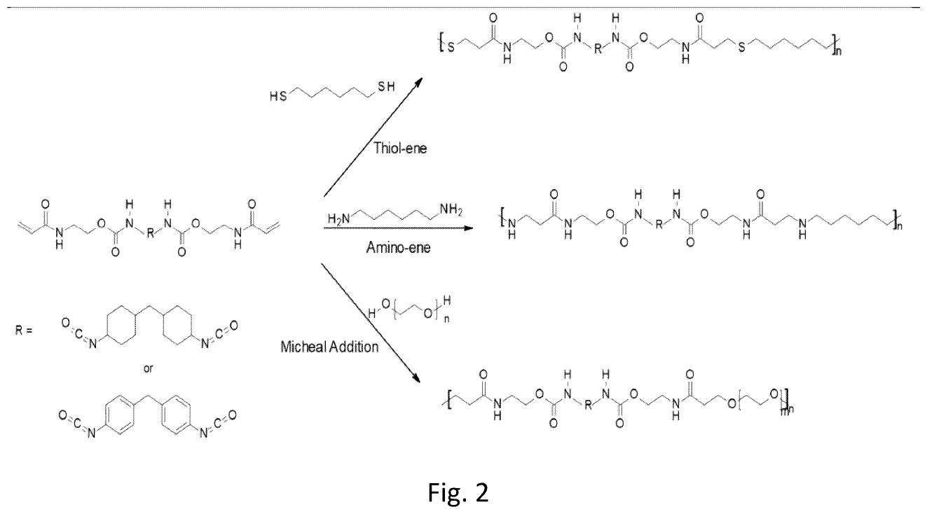

Further options for forming urethane-based acrylamides are those which employ different diisocyanates, including, among others, 4′-methylene bis(cyclohexyl isocyanate) and 4,4′-methylenebis(phenyl isocyanate) which are used to produce urethane-based acrylamides as those shown as reactants in FIG. 2. Although acrylamides having double carbon-carbon bonds are used in the above reactions, other unsaturated species having triple carbon-carbon bonds such as propiolamide groups may be used. The terminal unsaturated groups of the urethane-based acrylamides subsequently provide a platform to formulate polyurethanes in an open system without having concerns that any side reactions, such as urea formation, might occur. In particular, using the urethane-based acrylamide as a building block, it can be polymerized with three different functional groups: thiol, amine, and hydroxyl.

For example, the terminal alkyene groups of the urethane-based acrylamide may participate in a variety of reactions including various Michael addition reactions, including thiol-ene Michael addition reactions, amino-ene Michael addition reactions, and hydroxyl-ene Michael addition reactions.

For instance, in a thiol-ene Michael addition reactions, a urethane-based acrylamide precursor may be reacted with a polythiol precursor, HS—R′—SH, follows:

nH₂C═C—CO—NH—(CH₂)ₘ—O—CO—NH—R—NH—CO—O—(CH₂)ₘ—NH—CO—C═CH₂+nHS—R′—SH→ [symbol] S—CH₂—CH₂—CO—NH—(CH₂)ₘ—O—CO—NH—R—NH—CO—O—(CH₂)ₘ—NH—CO—CH₂—CH₂—S—R′ [symbol] ₙ

where n ranges from 1 to 100.

This reaction may be accelerated in the presence of a suitable catalyst. Different catalysts may be used for this purpose including base catalysts such as pentylamine and hexylamine (primary amines), triethylamine (tertiary amine), phosphines such as dimethylphenylphosphine (DMPP) and tris(2-carboxyethyl)phosphine (TCEP) , 1,8-Diazabicyclo[5.4.0]undec-7-ene (DBU) and 1,5-Diazabicyclo[4.3.0]non-5-ene (DBN). Reaction temperature may range, for example, from room temperature up to 50° C.

In an amino-ene Michael addition reaction, also called an amino-ene click reaction, a urethane-based acrylamide precursor may be reacted with a polyamine precursor, H₂N—R′—NH₂, thereby providing a polyamine urethane as follows:

nH₂C═C—CO—NH—(CH₂)ₘ—O—CO—NH—R—NH—CO—O—(CH₂)ₘ—NH—CO—C═CH₂+nH₂N—R′—NH₂→ [symbol] NH—CH₂—CH₂—CO—NH—(CH₂)ₘ—O—CO—NH—R—NH—CO—O—(CH₂)ₘ—NH—CO—CH₂—CH₂—NH—R′ [symbol] ₙ

where n ranges from 1 to 100. Reaction temperature may range, for example, from room temperature up to 50° C.

In a hydroxyl-ene Michael addition reaction, a urethane-based acrylamide precursor may be reacted with a polyol, HO—R′—OH, as follows:

nH₂C═C—CO—NH—(CH₂)ₘ—O—CO—NH—R—NH—CO—O—(CH₂)ₘ13 NH—CO—C═CH₂+nHO—R′—OH→ [symbol] O—CH₂—CH₂—CO—NH—(CH₂)ₘ—O—CO—NH—R—NH—CO—O—(CH₂)ₘ—NH—CO—CH₂—CH₂—O—R′ [symbol] ₙ

where n ranges from 1 to 100. Reaction temperature may range, for example, from room temperature up to 50° C.

In the above reactions R′ is an organic moiety, such as an aliphatic moiety or a polymeric moiety. For example, R′ may be a C₁-C₂₀ alkyl moiety. As another example, R′ may be a polymeric moiety selected from any of a variety of synthetic, natural, or hybrid synthetic-natural polymer moieties including, for example, polyether moieties including poly(alkylene oxides) such as (ethylene oxide) (PEO) (also referred to as polyethylene glycol or PEG) moieties, poly(propylene oxide) moieties, or poly(ethylene oxide-co-propylene oxide) moieties, polyphenylene oxide moieties, poly(N-vinyl pyrrolidone) moieties, vinyl alcohol polymer and copolymer moieties such as poly(vinyl alcohol) moieties, ethylene-vinyl alcohol copolymers moieties, polyoxazolines including poly(2-alkyl-2-oxazolines) moieties such as poly(2-methyl-2-oxazoline) moieties, poly(2-ethyl-2-oxazoline) moieties and poly(2-propyl-2-oxazoline) moieties, acrylic polymer moieties polymethyl methacrylate moieties, poly(hydroxyethyl methacrylate) moieties, poly(hydroxyethyl acrylate) moieties, acrylic copolymer moieties such as 2-hydroxyethyl methacrylate and methyl methacrylate copolymer moieties, poly(allyl alcohol) moieties, PEG methyl ether acrylate moieties, PEG methyl ether methacrylate moieties, PNIPAAM moieties, polysaccharide moieties, polyetherimide moieties, polyolefin moieties including polyethylene moieties, polypropylene moieties, polyisobutylene moieties, polybutadiene moieties, poly(4-methyl-1-pentene) moieties, and ethylene/propylene copolymer moieties, cyclic olefin copolymer moieties, halogenated polymer moieties such as polyvinyl chloride moieties and fluoropolymer moieties such as polytetrafluoroethylene moieties, polyvinylidene fluoride moieties, polyamide moieties such as nylon moieties and polyphthalamide moieties, styrene polymer and copolymer moieties such as polystyrene moieties and acrylonitrile butadiene styrene polymer moieties, copolymer moieties of styrene with olefin monomers such as isobutylene, isoprene and butadiene, for example polystyrene-polyisobutylene-polystyrene (SIBS) moieties, polystyrene-polyisoprene-polystyrene (SIS) moieties, polystyrene-polybutadiene-polystyrene (SBS) moieties, polycarbonate moieties, polyester poly such as polyethylene terephthalate moieties, polybutylene terephthalate moieties, polyoxymethylene (polyacetal) moieties, aliphatic polyketone moieties, aromatic polyketone moieties such as polyether ether ketones (PEEK) moieties, siloxane polymers moieties including poly(dimethyl siloxane) and dimethyl siloxane copolymer moieties, sulfone polymer moieties such as polysulfone moieties, polyethersulfone moieties, and polyphenylsulfone moieties, polyphenylene sulfide moieties, polyacrylonitrile moieties, polyimide moieties, polyetherimide moieties, bioabsorbable polymer moieties, such as poly(lactic acid) moieties, poly(glycolic acid) moieties, poly(ε-caprolactone) moieties, polyhydroxybutyrate valerate moieties, and bioabsorbable copolymer moieties such as poly(lactic acid-co-glycolic acid) moieties, among others.

In a thiol-ene Michael addition reaction in accordance with the present disclosure , a urethane-based acrylamide precursor as described herein may be reacted with a polythiol precursor (e.g., a dithiol precursor, a trithiol precursor or a higher polythiol precursor), for instance, an aliphatic dithiol, trithiol or higher polythiol precursor such as a C₁-C₂₀ alkyl dithiol, trithiol or higher polythiol precursor and/or may be reacted with any of a variety of synthetic, natural, or hybrid synthetic-natural polymer dithiol, trithiol or polythiol precursors including, for example, thiol-terminated polyethers including thiol-terminated poly(alkylene oxides) such as thiol-terminated poly(ethylene oxide) (PEO) (also referred to as polyethylene glycol or PEG), thiol-terminated poly(propylene oxide), thiol-terminated poly(tetramethylene oxide) or thiol-terminated poly(ethylene oxide-co-propylene oxide), and thiol-terminated polyphenylene oxide, thiol-terminated poly(N-vinyl pyrrolidone), thiol-terminated vinyl alcohol polymers and copolymers such as thiol-terminated poly(vinyl alcohol) and thiol-terminated ethylene-vinyl alcohol copolymers, thiol-terminated polyoxazolines including thiol-terminated poly(2-alkyl-2-oxazolines) such as thiol-terminated poly(2-methyl-2-oxazoline), thiol-terminated poly(2-ethyl-2-oxazoline) and thiol-terminated poly(2-propyl-2-oxazoline), thiol-terminated acrylic polymers such as thiol-terminated polymethyl methacrylate, thiol-terminated poly(hydroxyethyl methacrylate), thiol-terminated poly(hydroxyethyl acrylate), thiol-terminated acrylic copolymers such as thiol-terminated copolymers of 2-hydroxyethyl methacrylate and methyl methacrylate, thiol-terminated poly(allyl alcohol), thiol-terminated PEG methyl ether acrylate, thiol-terminated PEG methyl ether methacrylate, thiol-terminated PNIPAAM, thiol-terminated polysaccharides, thiol-terminated polyetherimide, thiol-terminated polyolefins including polyethylene, thiol-terminated polypropylene, thiol-terminated polyisobutylene, thiol-terminated polybutadiene, thiol-terminated poly(4-methyl-1-pentene), and thiol-terminated ethylene/propylene copolymers, thiol-terminated cyclic olefin copolymers, thiol-terminated halogenated polymers such as thiol-terminated polyvinyl chloride and thiol-terminated fluoropolymers such as thiol-terminated polytetrafluoroethylene, thiol-terminated polyvinylidene fluoride, thiol-terminated polyamides such as thiol-terminated nylons and thiol-terminated polyphthalamide, thiol-terminated styrene polymers and copolymers such as thiol-terminated polystyrene and thiol-terminated acrylonitrile butadiene styrene polymers, thiol-terminated copolymers of styrene with olefin monomers such as isobutylene, isoprene and butadiene, for example, thiol-terminated styrene-isobutylene-styrene (SIBS), thiol-terminated styrene-isoprene-styrene (SIS) copolymers, thiol-terminated styrene-butadiene-styrene (SBS) copolymers, thiol-terminated polycarbonate, thiol-terminated polyesters such as thiol-terminated polyethylene terephthalate, thiol-terminated polybutylene terephthalate, thiol-terminated polyoxymethylene (polyacetal), thiol-terminated aliphatic polyketones, thiol-terminated aromatic polyketones such as thiol-terminated polyether ether ketones (PEEK), thiol-terminated siloxane polymers including thiol-terminated poly(dimethyl siloxane) and thiol-terminated dimethyl siloxane copolymers, thiol-terminated sulfone polymers such as thiol-terminated polysulfone, thiol-terminated polyethersulfone, and thiol-terminated polyphenylsulfone, thiol-terminated polyphenylene sulfide, thiol-terminated polyacrylonitrile, thiol-terminated polyimides, thiol-terminated polyetherimide, thiol-terminated bioabsorbable polymers, such as thiol-terminated poly(lactic acid), thiol-terminated poly(glycolic acid), thiol-terminated poly(ε-caprolactone), thiol-terminated polyhydroxybutyrate valerate, thiol-terminated copolymers such as thiol-terminated poly(lactic acid-co-glycolic acid), among others.

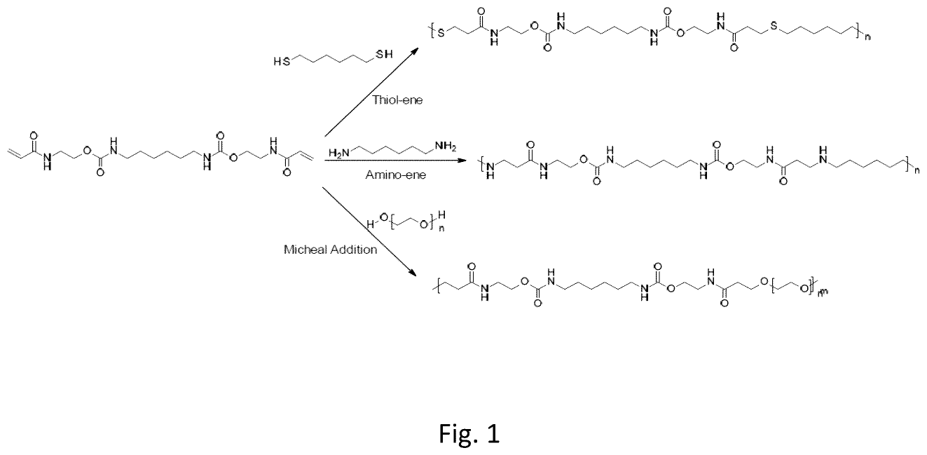

A specific thiol-ene Michael addition reaction in accordance with the present disclosure is shown in FIG. 1 wherein an aliphatic dithiol precursor, specifically, a C₁-C₂₀ alkyl dithiol, more specifically, 1,6-hexanedithiol, is reacted with a urethane-based acrylamide precursor, specifically,

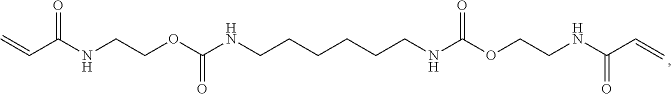 [SMILES: C=CC(=O)NCCOC(=O)NCCCCCCNC(=O)OCCNC(=O)C=C]

which may be formed from N-hydroxyethyl acrylamide and 1,6-diaminohexane as described above. Instead of or in addition to the aliphatic diol precursor, the reaction may also be conducted with a polymer thiol precursor, such as one of those described above. By carrying out the reaction with polymers of varying molecular weights, a range for properties may be afforded to the final product.

In an amino-ene Michael addition reaction in accordance with the present disclosure, a urethane-based acrylamide precursor as described herein may be reacted with a polyamine precursor (e.g., a diamine precursor, a triamine precursor or a higher polyamine precursor), for instance, an aliphatic diamine, triamine or higher polyamine precursor, such as a C₁-C₂₀ alkyl diamine, triamine or higher polyamine precursor, and/or may be reacted any of a variety of synthetic, natural, or hybrid synthetic-natural polymer diamine, triamine or higher polyamine precursor, including, for example, amino-terminated polyethers including amino-terminated poly(alkylene oxides) such as amino-terminated poly(ethylene oxide) (PEO) (also referred to as polyethylene glycol or PEG), amino-terminated poly(propylene oxide), amino-terminated poly(tetramethylene oxide) or amino-terminated poly(ethylene oxide-co-propylene oxide), and amino-terminated polyphenylene oxide, amino-terminated poly(N-vinyl pyrrolidone), amino-terminated vinyl alcohol polymers and copolymers such as amino-terminated poly(vinyl alcohol) and amino-terminated ethylene-vinyl alcohol copolymers, amino-terminated polyoxazolines including amino-terminated poly(2-alkyl-2-oxazolines) such as amino-terminated poly(2-methyl-2-oxazoline), amino-terminated poly(2-ethyl-2-oxazoline) and amino-terminated poly(2-propyl-2-oxazoline), amino-terminated acrylic polymers such as amino-terminated polymethyl methacrylate, amino-terminated poly(hydroxyethyl methacrylate), amino-terminated poly(hydroxyethyl acrylate), amino-terminated acrylic copolymers such as amino-terminated copolymers of 2-hydroxyethyl methacrylate and methyl methacrylate, amino-terminated poly(allyl alcohol), amino-terminated PEG methyl ether acrylate, amino-terminated PEG methyl ether methacrylate, amino-terminated PNIPAAM, amino-terminated polysaccharides, amino-terminated polyetherimide, amino-terminated polyolefins including polyethylene, amino-terminated polypropylene, amino-terminated polyisobutylene, amino-terminated polybutadiene, amino-terminated poly(4-methyl-1-pentene), and amino-terminated ethylene/propylene copolymers, amino-terminated cyclic olefin copolymers, amino-terminated halogenated polymers such as amino-terminated polyvinyl chloride and amino-terminated fluoropolymers such as amino-terminated polytetrafluoroethylene, amino-terminated polyvinylidene fluoride, amino-terminated polyamides such as amino-terminated nylons and amino-terminated polyphthalamide, amino-terminated styrene polymers and copolymers such as amino-terminated polystyrene and amino-terminated acrylonitrile butadiene styrene polymers, amino-terminated copolymers of styrene with olefin monomers such as isobutylene, isoprene and butadiene, for example, amino-terminated styrene-isobutylene-styrene (SIBS), amino-terminated styrene-isoprene-styrene (SIS) copolymers, amino-terminated styrene-butadiene-styrene (SBS) copolymers, amino-terminated polycarbonate, amino-terminated polyesters such as amino-terminated polyethylene terephthalate, amino-terminated polybutylene terephthalate, amino-terminated polyoxymethylene (polyacetal), amino-terminated aliphatic polyketones, amino-terminated aromatic polyketones such as amino-terminated polyether ether ketones (PEEK), amino-terminated siloxane polymers including amino-terminated poly(dimethyl siloxane) and amino-terminated dimethyl siloxane copolymers, amino-terminated sulfone polymers such as amino-terminated polysulfone, amino-terminated polyethersulfone, and amino-terminated polyphenylsulfone, amino-terminated polyphenylene sulfide, amino-terminated polyacrylonitrile, amino-terminated polyimides, amino-terminated polyetherimide, amino-terminated bioabsorbable polymers, such as amino-terminated poly(lactic acid), amino-terminated poly(glycolic acid), amino-terminated poly(ε-caprolactone), amino-terminated polyhydroxybutyrate valerate, amino-terminated copolymers such as amino-terminated poly(lactic acid-co-glycolic acid), among others.

A specific amino-ene Michael addition reaction in accordance with the present disclosure is shown in FIG. 1, wherein an aliphatic diamine precursor, specifically a C₁-C₂₀ alkyl diamine precursor, more specifically, 1,6-hexanediamine, is reacted with a urethane-based acrylamide, specifically,

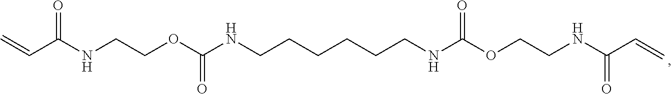 [SMILES: C=CC(=O)NCCOC(=O)NCCCCCCNC(=O)OCCNC(=O)C=C]

which may be formed from N-hydroxyethyl acrylamide and 1,6-diaminohexane as described above. Instead of or in addition to the aliphatic diamine precursor, the reaction may also be conducted with a polymer amine precursor, such as one of those described above. By carrying out the reaction with polymers of varying molecular weights, a range for properties may be afforded to the final product.

In a hydroxyl-ene Michael addition reaction in accordance with the present disclosure, a urethane-based acrylamide precursor as described herein may be reacted with a polyol precursor (e.g., a diol precursor, a triol precursor or a higher polyol precursor), for instance, an aliphatic diol, triol or higher polyol precursor, such as a C₁-C₂₀ alkyl diol, triol or higher polyol precursor, and/or may be reacted with any of a variety of synthetic, natural, or hybrid synthetic-natural polymer diol, triol or higher polyol precursors, including, for example, hydroxyl-terminated polyethers including hydroxyl-terminated poly(alkylene oxides) such as hydroxyl-terminated poly(ethylene oxide) (PEO) (also referred to as polyethylene glycol or PEG), hydroxyl-terminated poly(propylene oxide), hydroxyl-terminated poly(tetramethylene oxide) or hydroxyl-terminated poly(ethylene oxide-co-propylene oxide), and hydroxyl-terminated polyphenylene oxide, hydroxyl-terminated poly(N-vinyl pyrrolidone), hydroxyl-terminated vinyl alcohol polymers and copolymers such as hydroxyl-terminated poly(vinyl alcohol) and hydroxyl-terminated ethylene-vinyl alcohol copolymers, hydroxyl-terminated polyoxazolines including hydroxyl-terminated poly(2-alkyl-2-oxazolines) such as hydroxyl-terminated poly(2-methyl-2-oxazoline), hydroxyl-terminated poly(2-ethyl-2-oxazoline) and hydroxyl-terminated poly(2-propyl-2-oxazoline), hydroxyl-terminated acrylic polymers such as hydroxyl-terminated polymethyl methacrylate, hydroxyl-terminated poly(hydroxyethyl methacrylate), hydroxyl-terminated poly(hydroxyethyl acrylate), hydroxyl-terminated acrylic copolymers such as hydroxyl-terminated copolymers of 2-hydroxyethyl methacrylate and methyl methacrylate, hydroxyl-terminated poly(allyl alcohol), hydroxyl-terminated PEG methyl ether acrylate, hydroxyl-terminated PEG methyl ether methacrylate, hydroxyl-terminated PNIPAAM, hydroxyl-terminated polysaccharides, hydroxyl-terminated polyetherimide, hydroxyl-terminated polyolefins including polyethylene, hydroxyl-terminated polypropylene, hydroxyl-terminated polyisobutylene, hydroxyl-terminated polybutadiene, hydroxyl-terminated poly(4-methyl-1-pentene), and hydroxyl-terminated ethylene/propylene copolymers, hydroxyl-terminated cyclic olefin copolymers, hydroxyl-terminated halogenated polymers such as hydroxyl-terminated polyvinyl chloride and hydroxyl-terminated fluoropolymers such as hydroxyl-terminated polytetrafluoroethylene, hydroxyl-terminated polyvinylidene fluoride, hydroxyl-terminated polyamides such as hydroxyl-terminated nylons and hydroxyl-terminated polyphthalamide, hydroxyl-terminated styrene polymers and copolymers such as hydroxyl-terminated polystyrene and hydroxyl-terminated acrylonitrile butadiene styrene polymers, hydroxyl-terminated copolymers of styrene with olefin monomers such as isobutylene, isoprene and butadiene, for example, hydroxyl-terminated styrene-isobutylene-styrene (SIBS), hydroxyl-terminated styrene-isoprene-styrene (SIS) copolymers, hydroxyl-terminated styrene-butadiene-styrene (SBS) copolymers, hydroxyl-terminated polycarbonate, hydroxyl-terminated polyesters such as hydroxyl-terminated polyethylene terephthalate, hydroxyl-terminated polybutylene terephthalate, hydroxyl-terminated polyoxymethylene (polyacetal), hydroxyl-terminated aliphatic polyketones, hydroxyl-terminated aromatic polyketones such as hydroxyl-terminated polyether ether ketones (PEEK), hydroxyl-terminated siloxane polymers including hydroxyl-terminated poly(dimethyl siloxane) and hydroxyl-terminated dimethyl siloxane copolymers, hydroxyl-terminated sulfone polymers such as hydroxyl-terminated polysulfone, hydroxyl-terminated polyethersulfone, and hydroxyl-terminated polyphenylsulfone, hydroxyl-terminated polyphenylene sulfide, hydroxyl-terminated polyacrylonitrile, hydroxyl-terminated polyimides, hydroxyl-terminated polyetherimide, hydroxyl-terminated bioabsorbable polymers, such as hydroxyl-terminated poly(lactic acid), hydroxyl-terminated poly(glycolic acid), hydroxyl-terminated poly(ε-caprolactone), hydroxyl-terminated polyhydroxybutyrate valerate, hydroxyl-terminated copolymers such as hydroxyl-terminated poly(lactic acid-co-glycolic acid) among others.

A specific hydroxyl-ene Michael addition reaction in accordance with the present disclosure is shown in FIG. 1, wherein a diol polymer precursor, specifically, a hydroxyl-terminated PEG precursor, is reacted with a urethane-based acrylamide precursor, specifically,

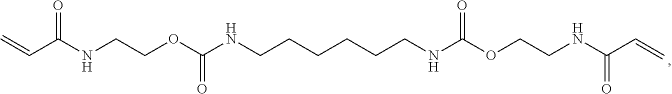 [SMILES: C=CC(=O)NCCOC(=O)NCCCCCCNC(=O)OCCNC(=O)C=C]

which may be formed from N-hydroxyethyl acrylamide and 1,6-diaminohexane as described above. By carrying out the reaction with polymers of varying molecular weights, a range for properties may be afforded to the final product. For example, carrying out the reaction with hydroxyl-terminated polyethylene glycols of varied molecular weights may offer a range of hardnesses for the product. Instead of or in addition to the diol polymer precursor, the reaction may also be conducted with an aliphatic diol, such as a C₁-C₂₀ alkyl diol.

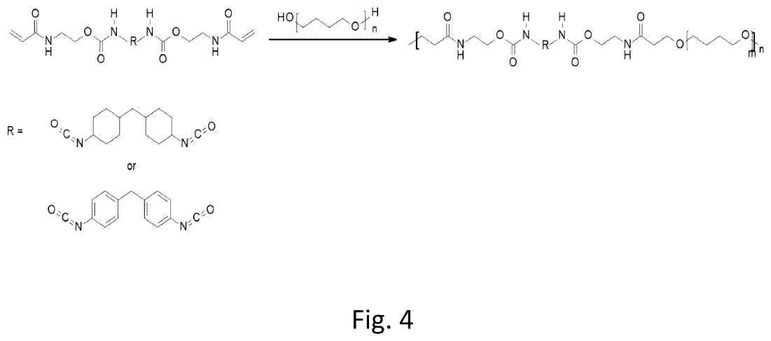

Further options for the reactions for forming urethane-based acrylamide precursors include, include those which employ different diisocyanates, including, among others, 4′-methylene bis(cyclohexyl isocyanate) (H12MDI) and 4,4′-methylenebis(phenyl isocyanate) (MDI) as shown in FIG. 2. Moreover, differing polythiols, polyamines and polyalcohols can be used in the above reactions for forming urethane-based acrylamides including thiol-terminated polyethers, thiol-terminated polycarbonates, thiol-terminated poly-epsilon-caprolactones, thiol-terminated polyisobutylenes, amino-terminated polyethers, amino-terminated polycarbonates, amino-terminated poly-epsilon-caprolactones, amino-terminated polyisobutylenes, other hydroxyl-terminated polyethers beyond PEG (such as hydroxyl-terminated poly(tetramethylene oxide) as shown in FIG. 4), hydroxyl-terminated polycarbonates, hydroxyl-terminated poly-epsilon-caprolactones, hydroxyl-terminated polyisobutylenes, among others describe above.

The novel polyurethanes described herein can be used to form a wide variety of polymeric regions for medical devices. These include polymeric regions that correspond to an entirety of a medical device, polymeric regions that correspond to medical device coatings, and polymeric regions that correspond to medical device components.

Such medical devices include the following, among others: leads, catheters such as balloon catheters, guide catheters, drug delivery catheters, guidewires such as percutaneous nephrolithotomy (PCNL) guidewires, occlusive devices such as Watchman® (mesh over nitinol), artificial heart valves, endoscopes, anchors, embolism coils, stents, ports, pumps, tubing, ventricular assist devices, filters, baskets, or drug delivery devices.

Depending on the particular polyurethane and the application, processing techniques that can be used include melt processing, solvent processing and thermal processing (e.g., where crosslinked networks are formed). Crosslinked networks are formed when the urethane-based acrylamide reactant has three, four, or more reactive unsaturated groups and/or where the other Michael additional reactant (e.g., an aliphatic-based reactant or a polymer-based reactant, particularly a multi-arm-polymer based reactant) has three, four or more thiol groups, three, four or more amino groups, or three or more hydroxyl groups.

Thus, numerous techniques can be used to form poly(urethane amide)-containing polymeric regions in accordance with the present invention.

For example, in some embodiments, thermoplastic processing techniques are used to form polymeric regions of various shapes and sizes. Using these techniques, polymeric regions can be formed by first providing a polymer melt that contains one or more poly(urethane amides) in accordance with the present disclosure, and one or more optional additives, as desired, and subsequently cooling the melt. Examples of thermoplastic techniques include compression molding, injection molding, blow molding, spinning, vacuum forming and calendaring, as well as extrusion into sheets, fibers, rods, tubes and other cross-sectional profiles of various lengths. Using these and other thermoplastic processing techniques, a variety of polymeric regions can be formed.

In some embodiments, solvent-based techniques are used to form the polymeric regions of various shapes and sizes. Using these techniques, polymeric regions can be formed by first providing a polymer solution that contains a solvent, one or more poly(urethane amides) in accordance with the present disclosure, and one or more optional additives, as desired, and subsequently removing the solvent. The solvent that is ultimately selected will contain one or more solvent species, which are generally selected based on their ability to dissolve the one or more poly(urethane amides) and any optional additives, as well as other factors, including drying rate, surface tension, etc. Examples of solvent-based techniques include solvent casting techniques, spin coating techniques, web coating techniques, solvent spraying techniques, dipping techniques, techniques involving coating via mechanical suspension including air suspension, ink jet techniques, electrostatic techniques, and combinations of these processes, among others.

In some embodiments, polymeric regions of various shapes and sizes may be formed from reactive precursor-containing fluids that contains (a) one or more urethane-based acrylamide precursors as described herein as well as (b) one or more Michael addition precursors selected from one or more dithiol, trithiol or higher polythiol precursors as described herein, one or more diamine, triamine or higher polyamine precursors as described herein, or one or more diol, triol or higher polyol precursors as described herein, (c) one or more suitable solvents, as needed and (d) one or more optional additives, as desired. Polymeric regions may be formed from such reactive liquids, for example using molding techniques, fiber spinning techniques, extrusion techniques, casting techniques, coating techniques, spraying techniques, laser-based 3-D printing techniques, and photo-based 3D printing, among others. In some embodiments, for example, where the precursors reactive relatively rapidly, the urethane-based acrylamide precursor(s) and Michael addition precursor(s) are not combined until immediately prior to molding, spinning, extrusion, casting, coating, or spraying.

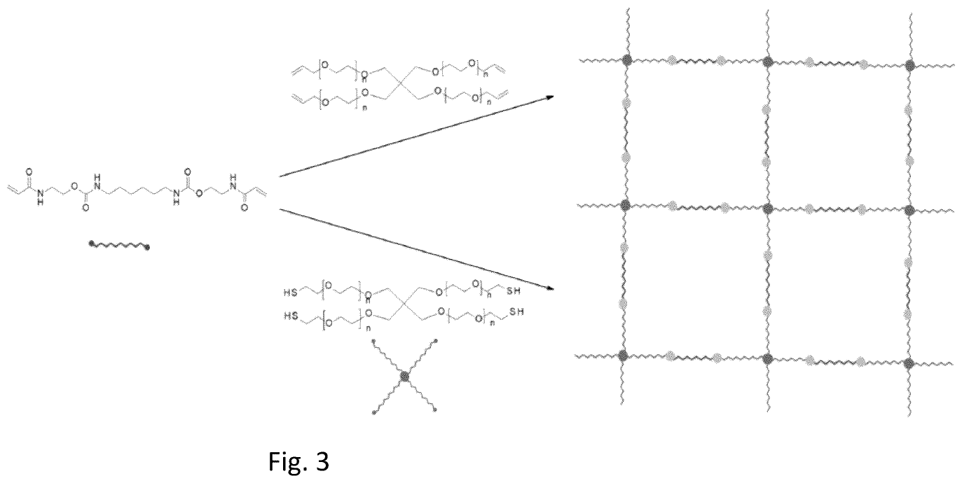

Thus, processing applications of the present technology include 3-D printing, which can be used to shape a wide variety of structures for use in medical devices. For example, as shown in FIG. 3, a crosslinked network may be formed by reacting one or more urethane-based acrylamide precursors as described herein with a Michael additional reactant, specifically, a multi-arm-polymer having four thiol groups. 3-D printing machines suitable for performing this process include digital light processing (DLP).

In some embodiments of the invention, a fluid, for example, a polymer melt, polymer solution or reactive precursor-containing fluid as described above, is applied to a substrate to form a polymeric region. For example, the substrate can correspond to all or a portion of an implantable or insertable medical device (e.g., one of those described above, among others) to which a polymeric region is applied. The substrate can also be, for example, a template, such as a mold, from which the polymeric region is removed after solidification. In other embodiments, for example, fiber spinning, extrusion and co-extrusion techniques, or particle spraying techniques, one or more polymeric regions are formed from the polymer melt, polymer solution or reactive precursor-containing without the aid of a substrate.

A variety of optional additives may be added to the polymeric regions of the present disclosure.

Such additives include one or more therapeutic agents, drug release modifiers, chemical stabilizers, leveling agents, colorants, plasticizers, wetting agents, catalysts, cross-linking agents, free radical initiators, lubricious agents, antioxidants, imaging agents including radiopaque agents, MRI contrast agents and echogenic agents.

Examples of therapeutic agents include, but are not limited to, antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters and drugs that may enhance neointimal formation such as the growth of endothelial cells.

Where a therapeutic agent is included, a wide range of therapeutic agent loadings can be used in conjunction with the medical devices of the present invention, with the therapeutically effective amount being readily determined by those of ordinary skill in the art and ultimately depending, for example, upon the condition to be treated, the age, sex and condition of the subject, the nature of the therapeutic agent, the nature of the polymeric region(s), and the nature of the medical device, among other factors.

1. A medical device, at least a portion of which comprises a polymeric region comprising a poly(urethane amide) that comprises urethane (—O—CO—NH—) groups, and amide (—CO—NH—) groups.

2. The medical device of claim 1, wherein the poly(urethane amide) further comprises amine (—NH—) linkages.

3. The medical device of claim 1, wherein the poly(urethane amide) further comprises thioether (—S—) linkages.

4. The medical device of claim 1, wherein the poly(urethane amide) comprises (a) one or more diisocyanate residues selected from C₆-C₂₀ aromatic diisocyanate residues, C₄-C₂₀ aliphatic diisocyanate residues, and C₆-C₂₀ cycloaliphatic diisocyanate residues, (b) residues of a C₄-C₁₀ hydroxyalkyl acrylamide, and (c) residues of a polythiol, a polyamine, or a polyol.

5. The medical device of claim 4, wherein the one or more diisocyanate residues are selected from residues of toluene diisocyanate (TDI), methylene diphenyl diisocyanate (MDI), naphthalene diisocyanate (NDI), toluidine diisocyanate, 1,6-hexamethylene diisocyanate (HDI), tetramethylene diisocyanate, 1-isocyanato-3-isocyanatomethyl-3,5,5-trimethyl-cyclohexane (isophorone diisocyanate) or 4,4′-diisocyanato dicyclohexylmethane (H₁₂MDI).

6. The medical device of claim 4, wherein the polythiol is selected from thiol terminated C₆-C₂₀ aliphatic molecules, thiol terminated polyethers, thiol terminated polycarbonates, thiol terminated poly-epsilon-caprolactones, thiol terminated polyolefins, thiol terminated polyesters, thiol terminated polyoxazolines and thiol terminated polyvinylpyrrolidone.

7. The medical device of claim 4, wherein the polyol is selected from hydroxyl terminated C₆-C₂₀ aliphatic molecules, hydroxyl terminated polyethers, hydroxyl terminated polycarbonates, hydroxyl terminated poly-epsilon-caprolactones, hydroxyl terminated polyolefins, hydroxyl terminated polyesters, hydroxyl terminated polyoxazolines and hydroxyl terminated polyvinylpyrrolidone.

8. The medical device of claim 4, wherein the polyamine is selected from amino terminated C₆-C₂₀ aliphatic molecules, amino terminated polyethers, amino terminated polycarbonates, amino terminated poly-epsilon-caprolactones, amino terminated polyolefins, amino terminated polyesters, amino terminated polyoxazolines and amino terminated poly vinyl pyrrolidone.

9. The medical device of claim 1, wherein the poly(urethane amide) is of one of the following formulas (I), (II), or (III): [symbol]
S—CH₂—CH₂—CO—NH—(CH₂)ₘ—O—CO—NH—R—NH—CO—O—(CH₂)ₘ—NH—CO—CH₂—CH₂—S—R′ [symbol] ₙ (I) [symbol]
NH—CH₂—CH₂—CO—NH—(CH₂)ₘ—O—CO—NH—R—NH—CO—O—(CH₂)ₘ—NH—CO—CH₂—CH₂—NH—R′ [symbol] ₙ (II) [symbol]
O—CH₂—CH₂—CO—NH—(CH₂)ₘ—O—CO—NH—R—NH—CO—O—(CH₂)ₘ—NH—CO—CH₂—CH₂—O—R′ [symbol] ₙ (III)
wherein m is an integer ranging from 1 to 20, n is an integer ranging from 1 to 1000, R is selected from C₂-C₂₀ aliphatic moieties, C₆-C₂₀ aromatic moieties, and C₆-C₂₀ cycloaliphatic moieties, and wherein R′ is selected from C₂-C₂₀ aliphatic moieties, polyether moieties, polycarbonate moieties, poly-epsilon-caprolactone moieties, polyolefin moieties, polyester moieties, polyoxazoline moieties and polyvinyl pyrrolidone moieties.

10. The medical device of claim 9, wherein the poly(urethane amide) of the formula (I) is formed by reaction between H₂C═C—CO—NH—(CH₂)ₘ—O—CO—NH—R—NH—CO—O—(CH₂)ₘ—NH—CO—C═CH₂ and HS—R′—SH.

11. The medical device of claim 9, wherein the poly(urethane amide) of the formula (II) is formed by reaction between H₂C═C—CO—NH—(CH₂)ₘ—O—CO—NH—R—NH—CO—O—(CH₂)ₘ—NH—CO—C═CH₂ and H₂N—R′—NH₂.

12. The medical device of claim 9, wherein the poly(urethane amide) of the formula (III) is formed by reaction between H₂C═C—CO—NH—(CH₂)ₘ—O—CO—NH—R—NH—CO—O—(CH₂)ₘ—NH—CO—C═CH₂ and HO—R′—OH.

13. The medical device of claim 9, wherein R is selected from a benzene moiety, a toluene moiety, a diphenyl methylene moiety, a naphthalene moiety, a hexamethylene moiety, a tetramethylene moiety, an isophorone moiety and a dicyclohexyl methylene moiety.

14. The medical device of claim 1, wherein the polymeric region corresponds to an entirety of the medical device.

15. The medical device of claim 1, wherein the polymeric region corresponds to a medical device coating.

16. The medical device of claim 1, wherein the polymeric region corresponds to a medical device component.

17. The medical device of claim 1, wherein the polymeric region further comprises a therapeutic agent.

18. The medical device of claim 1, wherein the polymeric region further comprises an additive.

19. A method of making a medical device comprising:
(a) performing a Michael addition reaction between H₂C═C—CO—NH—(CH₂)ₘ—O—CO—NH—R—NH—CO—O—(CH₂)ₘ—NH—CO—C═CH₂ and HS—R′—SH to form a poly(urethane amide of the formula (I): [symbol]
S—CH₂—CH₂—CO—NH—(CH₂)ₘ—O—CO—NH—R—NH—CO—O—(CH₂)ₘ—NH—CO—CH₂—CH₂—S—R′ [symbol] ₙ (I);
(b) performing a Michael addition reaction between H₂C═C—CO—NH—(CH₂)ₘ—O—CO—NH—R—NH—CO—O—(CH₂)ₘ—NH—CO—C═CH₂ and H₂N—R′—NH₂ to form a poly(urethane amide of the formula (II): [symbol]
NH—CH₂—CH₂—CO—NH—(CH₂)ₘ—O—CO—NH—R—NH—CO—O—(CH₂)ₘ—NH—CO—CH₂—CH₂—NH—R′ [symbol] ₙ (II); or
(c) performing a Michael addition reaction between H₂C═C—CO—NH—(CH₂)ₘ—O—CO—NH—R—NH—CO—O—(CH₂)ₘ—NH—CO—C═CH₂ and HO—R′—OH to form a poly(urethane amide of the formula (III): [symbol]
O—CH₂—CH₂—CO—NH—(CH₂)ₘ—O—CO—NH—R—NH—CO—O—(CH₂)ₘ—NH—CO—CH₂—CH₂—O—R′ [symbol] ₙ (III);
wherein m is an integer ranging from 1 to 20, n is an integer ranging from 1 to 1000, R is selected from C₂-C₂₀ aliphatic moieties, C₆-C₂₀ aromatic moieties, and C₆-C₂₀ cycloaliphatic moieties, and wherein R′ is selected from C₂-C₂₀ aliphatic moieties, polyether moieties, polycarbonate moieties, poly-epsilon-caprolactone moieties, polyolefin moieties, polyester moieties, polyoxazoline moieties and polyvinyl pyrrolidone moieties.

20. A 3-D printing process for forming a medical device or a medical device component comprising reacting (a) a urethane-based acrylamide reactant that comprises a residue of a C₄-C₁₀ hydroxyalkyl acrylamide and a diisocyanate residue selected from a C₆-C₂₀ aromatic diisocyanate residue, a C₄-C₂₀ aliphatic diisocyanate residue, and a C₆-C₂₀ cycloaliphatic diisocyanate residue and (b) a multi-arm polymer comprising 3 or more polymer arms and three or more terminal groups selected from thiol groups, amino groups and hydroxyl groups.
